SEQUENCE LISTING (7 sequences)

SEQ ID NO: 1 - protein, 102 aa, Homo sapiens
MHSLFVRHPDFKTRLGLSAALPFPSCVTLDEIFGHSIAQSSICKIRESNCLHCVDNRDGKPLLRQQARVMAAFSSLLFTSGTWLMVLISCPCTLTVSFCGHL

SEQ ID NO: 2 - protein, 129 aa, Homo sapiens [sequence omitted]

SEQ ID NO: 3 - protein, 29 aa, Homo sapiens
MHSLFVRHPDFKTRLGLSAALPFPSCVTL

SEQ ID NO: 4 - DNA, 309 bp, Homo sapiens
atgcacagcttattcgtcaggcacccagacttcaaaaccagactaggattgagtgcagccctgccatttcccagctgtgtgaccttggatgaaatatttggccactctatagcccagtcatcaatctgtaaaattagggagagtaattgtctccactgtgtagataacagagatggcaagcctttgttgcgccagcaggccagagtcatggctgccttttcttctctgctcttcacgtcggggacatggctgatggtcttaatttcatgcccttgcacactgactgtcagtttttgtggacatctgtag

SEQ ID NO: 5 - DNA, 423 bp, Homo sapiens
atgtttgcctcccacacacctaatttgacctctaccccaccccagcctactaatgctggctacagatgtccacaaaaactgacagtcagtgtgcaagggcatgaaattaagaccatcagccatgtccccgacgtgaagagcagagaagaaaaggcagccatgactctggcctgctggcgcaacaaaggcttgccatctctgttatctacacagtggagacaattactctccctaattttacagattgatgactgggctatagagtggccaaatatttcatccaaggtcacacagctgggaaatggcagggctgcactcaatcctagtctggttttgaagtctgggtgcctgacgaataagctgtgcatttttcaagagaaggccttctagagagggtgtgactttggtactcttgtcctgtga

SEQ ID NO: 6 - DNA, 751 bp, Homo sapiens
ttttcacaggacaagagtaccaaagtcacaccctctctagaaggccttctcttgaaaaatgcacagcttattcgtcaggcacccagacttcaaaaccagactaggattgagtgcagccctgccatttcccagctgtgtgaccttggatgaaatatttggccactctatagcccagtcatcaatctgtaaaattagggagagtaattgtctccactgtgtagataacagagatggcaagcctttgttgcgccagcaggccagagtcatggctgccttttcttctctgctcttcacgtcggggacatggctgatggtcttaatttcatgcccttgcacactgactgtcagtttttgtggacatctgtagccagcattagtaggctggggtggggtagaggtcaaattaggtgtgtgggaggcaaacatgctaaagagggtcaaacccgccacatcattttatttctttgtgggaagcagcaacagtacctcggttcctaaggtctttctggagctgtggttctccaaaaaacattccagattttacccctcaggggtcatctggcaatgtctggagatacatttggttgtggcaacatgaggagggaagggttgtcactagtgtctagtgggtgtgggccagggatgctgcttagcagcctacatacagcacagagggcagtcccctcaacagagaatgattcggttgcaaatgtcaactgtggcgaggtcgggaaaccctgtctggtgtgag

SEQ ID NO: 7 - protein, 5 aa, Homo sapiens
GSAAL

(19) United States
(12) Patent Application Publication
(10) Pub. No.: US 2003/0119044 A1
(43) Pub. Date: Jun. 26, 2003

(54) Tumor suppressors associated with human chromosome 21q22

(75) Inventor: Markus H. Frank, Cambridge, MA (US)

Correspondence Address: Michael A. Sanzo, Fitch, Even, Tabin & Flannery, Suite 401L, 1801 K Street, N.W., Washington, DC, 20006-1201, US

(73) Assignee: The Brigham and Women's Hospital, Inc.

(21) Appl. No.: 10/303,995

(22) Filed: Nov. 26, 2002

Related U.S. Application Data
(60) Provisional application No. 60/334,056, filed on Nov. 30, 2001.

(51) Int. Cl. C12Q 1/68; C07H 21/04; C12P 21/02; C12N 5/06; C07K 14/47

(52) U.S. Cl. 435/6; 435/69.1; 435/320.1; 435/325; 530/350; 536/23.2

(57) ABSTRACT

The present invention is directed to tumor suppressor polypeptides and to polynucleotide sequences by which they are encoded. It includes assays designed for detecting mutations that predispose a patient to certain forms of cancer and therapeutic methods in which suppressor proteins or genes encoding the proteins are administered to patients.

### CROSS REFERENCE TO RELATED APPLICATION

The present application claims the benefit of U.S. provisional application No. 60/334,056, filed on Nov. 30, 2001.

### FIELD OF THE INVENTION

The present invention is directed to tumor cell suppressors and polynucleotides encoding the suppressors. Mutations in these polynucleotides predispose a patient to the development of cancer, particularly multiple myeloma. In addition, the invention is directed to methods of treating patients by administering either suppressor proteins or polynucleotides encoding the proteins.

### BACKGROUND OF THE INVENTION

Allelic imbalances including loss of heterozygosity (LOH) and microsatellite instability (MI) on the long arm of chromosome 21 (21q) have been found in several types of human cancer. Putative tumor suppressor loci have been mapped to 21q22 in multiple myeloma, acute myeloid leukemia, myelodysplastic syndrome, carcinoma of the gallbladder, primary gastric cancers and oral squamous cell carcinoma, among others (Bergsagel, et al., Proc. Nat'l Acad. Sci. USA 93:13931-13936 (1996): Ferrari, et al., Ann. Hematol. 80:72-73 (2001): Gao et al., Genes Chromosomes Cancer 28:164-172 (2000); Nakayama, et al., Cancer Lett. 166:135-141 (2001); Park, et al., Cancer Lett. 159:15-21 (2000); Yamamoto, et al., Oncol. Rep. 6:1223-1227 (1999)). In addition, the 21q22 chromosomal region harbors the Down syndrome critical region for acute myeloid leukemia (AML) which is a hematological malignancy occurring at increased frequency in children afflicted with Down syndrome (trisomy 21). These findings suggest that allelic imbalances on 21q22 may be involved in the development of a large variety of human cancers.

In multiple myeloma, one such genetic imbalance has been defined on the molecular level and the breakpoint junction for a t(14;21) (q32;q22) translocation has been sequenced (Bergsagel, et al., Proc. Nat'l Acad. Sci. USA 93:13931-13936 (1996); GenBank Accession No. U73676). The further investigation of genes associated with multiple myeloma and similar cancers may lead to better methods of diagnosing patients at an early stage of disease development and to better methods of treatment.

### SUMMARY OF THE INVENTION

The present invention is based upon the identification of a novel gene located on human chromosome 21q22 in the Down syndrome critical region. The gene consists of two distinct complete coding segments, each directly encompassing the reported multiple myeloma t(14;21) (32;q22) translocation breakpoint junction. The expression of the 21q22 gene is specifically impaired in certain forms of multiple myeloma, pointing to a direct roll in the pathogenesis of these tumors.

An examination in the structure of the new gene indicates that it encodes two distinct proteins. The protein of SEQ ID NO: 1 is encoded by a single exon (SEQ ID: 4) of the sense human genomic sequence. It encompasses one C-terminal transmembrane domain and a 29 amino acid N-terminal cleavable peptide (SEQ ID NO: 3). PSORT II analysis suggests a subcellular localization of the protein in vesicles of the secretory system. The cleavable peptide (SEQ ID NO: 3), contains a N-myristoylation motif, G-S-A-A-L (SEQ ID NO: 7), also suggesting that either the protein of SEQ ID NO: 1 or its cleavable peptide may be secreted. The protein of SEQ ID NO: 2 is encoded by a single exon (SEQ ID NO: 5) on the antisense strand of human genomic DNA, encompasses no transmembrane domains and is predicted to have a nuclear subcellular localization that plays a role in regulating cell growth.

In its first aspect, the invention is directed to isolated or substantially pure proteins or peptides consisting essentially of the amino acid sequences of SEQ ID NO: 1; SEQ ID NO: 2; or SEQ ID NO: 3. The term “consisting essentially of” is meant to encompass polypeptides having exactly the same amino acid sequence at those in the Sequence Listing as well as proteins with differences that are not substantial as evidenced by their retaining the functional properties of the tumor suppressor proteins. More specifically, a protein will consist essentially of the amino acid sequence of SEQ ID NO: 1, SEQ ID NO: 2 or SEQ ID NO: 3 if it is at least seventy percent structurally identical and it maintains essentially the same biological activity. Preferably, proteins will have eighty or ninety percent structural identity. An analogous definition applies with respect to polynucleotides. The terms “substantially pure” and “isolated” refer to polypeptides or polynucleotides that have been separated from other accompanying biological components. Substantially purified or isolated molecules will constitute at least eighty percent of a sample, with greater percentages being preferred. Many means are available for assessing the purity of a protein or nucleic acid within a sample, including analysis by polyacrylamide gel electrophoresis, chromatography and analytical centrifugation. Western blocks may also be used to assess purity using antibodies directed against specific epitopes of a protein.

The invention is also directed to isolated polynucleotides that encode the polypeptides described above. Specific polynucleotide sequences consist essentially of the sequences of SEQ ID NO: 4; SEQ ID NO: 5; or SEQ ID NO: 6. In addition, the invention includes vectors comprising the polynucleotides and, especially, expression vectors in which the polynucleotide constitutes a structural coding region which is operably linked to a promoter. The term “operably linked” indicates that the transcription of the coding region is under the control of the promoter and that protein having the correct sequence is eventually produced.

In addition, the invention includes host cells transformed with the vectors described above. These host cells may take several different forms. For example, they may constitute either bacterial, or preferably, mammalian cells designed to produce tumor suppressor polypeptide in vitro. Alternatively, they may constitute cells that have been removed from a patients body and transformed with the intention of being reimplanted for the in vivo production of suppressor. Finally, host cells may be produced by transforming cells directly in vivo as part of a therapeutic regimen.

In another aspect, the invention is directed to an assay for determining whether a subject either has, or has a predisposition for developing, cancer, particularly multiple myeloma. A cell or tissue sample is removed from the subject and a determination is made whether mutations have occurred in genomic regions encoding the polypeptides of SEQ ID NO: 1, SEQ ID NO: 2, or SEQ ID NO: 3. Mutational analysis may be carried out by any method known in the art, although PCR amplification followed by sequence analysis is generally preferred. Primers for PCR can be generated from the sequence information as presented herein. Of particular interest are mutations that result in a loss of biological activity of encoded proteins. For example, mutations that resulted in an inability of protein to retard the growth of myeloma cells are particularly important. Initially, it may be necessary to test polypeptides having mutations to determine the extent to which they are secreted and limit myeloma cell growth. Once specific mutations have been identified as leading to a loss of activity, biological assays will no longer be essential.

The invention is also directed to methods of treating patients having cancer, and particularly multiple myeloma, by administering any of the tumor suppressors described above, i.e., the polypeptides of SEQ ID NO: 1, SEQ ID NO: 2 or SEQ ID NO: 3. Administration may be accomplished by the direct parenteral delivery of polypeptide, or, alternatively, by administering a polynucleotide encoding the suppressor. Methods for the in vivo delivery of nucleic acids are well known in the art and may involve the use of viral vectors (adenoviral vectors), chemical agents promoting in vivo cell transformation (e.g., liposomes), or the direct administration of naked DNA. Alternatively, cells may be removed from a patient, transformed and then reimplanted. In addition to being useful for the treatment of cancer, it is believed that these procedures will also be of value in treating other diseases characterized by abnormal cellular proliferation, autoimmune diseases such as multiple sclerosis and inflammatory diseases such as rheumatoid arthritis.

### DETAILED DESCRIPTION OF THE INVENTION

The present invention is directed to a novel 21q22 gene that encodes tumor suppressor proteins which have structural and biological characteristics that indicate that they are secreted from cells and act in an autocrine/paracrine matter to control cell growth. Disruption of the gene leads to a loss of growth control, thereby leading to the development of certain cancers, particularly multiple myeloma. The invention includes assays for detecting mutations that would lead to a loss of growth control and to therapies aimed at replenishing the intact gene using gene therapy methods or by directly administering polypeptides to patients.

### I. Protein Nucleic Acid Sequences

The structure of the tumor suppressor proteins of the present invention are shown in SEQ ID NO: 1 and SEQ ID NO: 2. These proteins are encoded by the polynucleotides shown as SEQ ID NO: 4 and SEQ ID NO: 5. In addition, there is a 29 amino acid peptide that is cleaved from the protein of SEQ ID NO: 1 which may also be used as a therapeutic agent and which is shown as SEQ ID NO: 3. Finally, SEQ ID NO: 6 is the entire gene and mRNA from which from which SEQ ID NO: 4 and SEQ ID NO: 5 were derived. It will be understood that the invention encompasses not only sequences identical to those shown but also sequences that are essentially the same as evidenced by their retaining the same basic structural and functional characteristics. For example, techniques such site directed mutagenesis may be used to introduce variations into a proteins structure. Variations in protein structure introduced by this or other similar methods are encompassed by the invention provided that the resulting polypeptide retains its biological characteristics, particularly with respect to controlling cell growth.

Many methods are available for producing and isolating DNA or proteins such as those described herein (see e.g., Sambrook, et al., Molecular Cloning: A Laboratory Manual, 2^{nd} ed., Cold Spring Harbor Press (1989)). For example, one method is to screen a cDNA library that has been prepared by reverse transcribing mRNA isolated from tissues or cells that express the gene. The library may be screened using probes synthesized based upon the sequences shown in the Sequence Listing. Alternatively, amplification of the desired sequences may be achieved using the polymerase chain reaction (“PCR”) of reverse transcribed RNA. Primers for PCR may be constructed using the sequences shown in SEQ ID NO: 4, SEQ ID NO: 5 or SEQ ID NO: 6. Confirmation that the correct sequence has been amplified may be obtained by sequencing amplification products. Finally, the sequences shown are relatively short in nature and both nucleic acids and proteins may be obtained using standard synthetic methods.

### II. Production of Suppressor Protein

Apart from the chemical synthesis of protein or peptide, production may, if desired, be carried out by recombinant means. Expression may be induced in a host cell by transforming it with an appropriate expression vector. The vector should contain transcriptional and translational signals recognizable by the host, together with the desired structural sequence in an operable linkage, i.e., nucleotides encoding a suppressor protein should be positioned such that regulatory sequences present in the vector control the synthesis of mRNA and a protein having the correct sequence is ultimately produced.

Preferably, nucleic acid encoding suppressor protein is expressed in eukaryotic cells, especially mammalian cells. Such cells are capable of promoting post-translational modifications necessary to ensure that the recombinant protein is structurally and functionally the same as that found in nature. Examples of mammalian cells known to provide post-translational modification of cloned proteins include inter alia, NIH-3T3 cells, CHO cells, HeLA cells, LM (tk-) cells, and the like. Eukaryotic promoters known to control recombinant gene expression are preferably utilized and may include that of the mouse metallothionein I gene, the TK promoter of Herpes virus, the CMV early promoter and the SV40 early promoter.

Expression vectors may be introduced into host cells by any method known in the art (e.g., calcium phosphate precipitation, microinjection, electroporation, or viral transfer) and cells expressing recombinant protein can be selected by established techniques. Confirmation of expression may be obtained by PCR amplification using primers selected from the sequences shown in SEQ ID NO: 4, SEQ ID NO: 5 or SEQ ID NO: 6.

Recombinant protein may be purified using standard techniques well known in the art. Such techniques may include filtration, precipitation, chromatography and electrophoretic methods. Purity can be assessed by performing electrophoresis on a polyacrylamide gel and visualizing proteins using standard staining methodology.

### III. Antibodies to Tumor Suppressor Polypeptides

The present invention is also directed to antibodies raised against the tumor suppressor proteins of SEQ ID NO: 1, SEQ ID NO: 2 or SEQ ID NO: 3. The process for producing such antibodies may involve injecting protein into an appropriate animal or injecting short antigenic peptides made to correspond to different regions of the protein. These peptides should be at least 5 amino acids in length and should, preferably, be selected from regions believed to be unique to the tumor suppressor protein. Methods for generating and detecting antibodies are well know in the art and are taught by such references as: Harlow, et al., Antibodies, A Laboratory Manual, Cold Spring Harbor Laboratory, N.Y. (1988); Klein, Immunology: The Science of Self-Nonself Discrimination, (1982); and Campbell, “Monoclonal Antibody Technology”, in Laboratory Techniques in Biochemistry and Molecular Biology, (1984).

The term “antibody”, as used herein, is meant to include intact molecules as well as fragments that retain their ability to bind antigen, such as Fab and F(ab′)₂ fragments. The term “antibody” is also defined as referring to both monoclonal antibodies and polyclonal antibodies. Polyclonal antibodies are derived from the sera of animals immunized with a tumor suppressor antigen. Monoclonal antibodies to a suppressor can be prepared using hybridoma technology, as taught by such references as: Hammerling, et al., in: Monoclonal Antibodies and T-Cell Hybridomas, Elsevier, N.Y., pp. 563-681 (1981). In general, this technology involves immunizing an immunocompetent animal, typically a mouse, with either intact protein, or a fragment derived therefrom. Splenocytes are then extracted from the immunized animal and are fused with suitable myeloma cells, such as SP₂O cells. Thereafter, the resulting hybridoma cells are selectively maintained in HAT medium and then cloned by limited dilution (Wands, et al., Gastroenterology 80:225-232 (1981)). Cells obtained through such selection are then assayed to identify clones which secrete antibodies capable of binding suppressor protein.

Antibodies or fragments of antibodies of the invention may be used to detect the presence of tumor suppressor protein in any of a variety of immunoassays. These assays may be useful in determining whether a particular individual shows abnormally low levels of suppressor protein and may therefore be susceptible to the development of conditions characterized by unregulated cell growth. For example, antibodies may be used in radioimmunoassays or in immunometric assays, also known as “two-site” or “sandwich” assays. In a typical immunometric assay, a quantity of unlabeled antibody is bound to a solid support that is insoluble in the fluid being tested. Following the initial binding of antigen to immobilized antibody, a quantity of detectably labeled second antibody (which may or may not be the same as the first) is added to permit detection and/or quantitation of bound antigen (see, e.g. Radioimmune Assay Method, Kirkham, et al., ed. pp. 199-206, E&S Livingstone, Edinburgh (1970)). Many variations of these types of assays are known in the art and may be employed for the detection of suppressor protein.

Antibodies to protein may also be used in purification procedures (see generally, Dean et al., Affinity Chromatography, A Practical Approach, IRL Press (1986)). Typically, these procedures involve immobilizing antibody on a chromatographic matrix such as Sepharose, 4B. The matrix is then packed into a column and the preparation containing suppressor protein is passed through under conditions that promote binding, e.g., under low salt conditions. The column is then washed and protein is eluted using a buffer that promotes dissociation from antibody, e.g., in a buffer having an altered pH or salt concentration. The eluted protein may be transferred into a buffer, for example via dialysis, and thereafter either stored or used directly.

Antibodies may also be used in Western blotting for the detection of suppressor protein in a sample. Shifts in the position of bands obtained in Western blots may be indicative of mutations in a protein.

### IV. Assays for Mutated Genes

Many methods are available for detecting mutations in the genes encoding tumor suppressor proteins. Preferably, sequences are amplified using primers that flank the region being analyzed. The amplification product may then be either sequenced using standard methods or analyzed electrophoretically to determine its size. Changes in sequence or size are indicative of a mutation that may effect the activity of the encoded protein. In order to determine the seriousness of the mutation, the mutated protein may be synthesized and then analyzed to determine its ability to suppress the growth of myeloma cells.

Mutations may also be detected using immunoassays of the type discussed above. Mutations may be reflected either in a loss of detectable protein relative to that seen in normal individuals or by a change in protein size, e.g., detected using Western blot. The antibodies may also be used to purify proteins by affinity methods for direct structural analysis.

### V. Therapeutic Methods

Therapeutic methods may involve either the administration of polypeptide or the administration of nucleic acids that encode the polypeptide. In the latter case, oligonucleotides designed for the expression of suppressor may be administered directly to patients or, alternatively, cells from patients may be removed, transfected and then reimplanted. The in vivo transfection of cells has been known for many years and may be accomplished using viral vectors (see e.g. U.S. Pat. No. 6,020,191); liposomes (see e.g., Nicolau, Meth. Enzymol 149:157-176 (1987)); DNA complexed to agents that facilitate cellular uptake (see e.g., U.S. Pat. No. 5,264,618; WO 98/14431); or even by simply injecting naked DNA (see e.g., U.S. Pat. No. 5,693,622). Administration may be repeated as is necessary until a positive therapeutic effect is observed. For example, DNA may be administered to a patient until the growth of cancer cells is retarded or until tumors diminish in size. Administration may be continued thereafter based upon clinical considerations.

As an alternative to gene therapy, suppressor polypeptide may be directly administered to a patient in order to preserve activity. In general, the polypeptide should be administered parentally, with administration by injection being preferred. The dosage administered to a patient will be determined by the attending physician based upon clinical considerations and using methods well known in the art.

Suppressor polypeptides may be administered in either a single or multiple dosage regimen and may be given either alone or in conjunction with other therapeutic agents. Parenteral compositions may be used for intravenous, intraarterial, intramuscular, intraperitoneal, intracutaneous, or subcutaneous delivery. These preparations may be made using conventional techniques and may include isotonic saline, water, polyglycols, Ringer's solution, etc. Topical compositions may also be useful in treating cancers of the skin. All dosage forms may be prepared using methods that are standard in the art (see e.g., Remington's Pharmaceutical Sciences, 16 edition, A. Oslo editor, Easton, Pa. (1980)).

### EXAMPLES

In multiple myeloma, the t(14;21) (q32;q22) translocation is a genetic imbalance which has previously been defined on the molecular level and the breakpoint junction has been sequenced. Since this genomic region is believed to harbor a tumor suppressor gene, PCR primers were designed based on sequence information available in the database at the National Center for Biotechnology Information (NCBI) to amplify this putative gene locus for the purpose of detecting hitherto unknown genes. Using these gene-specific oligonucleotide primers and the PCR technique on reverse transcribed total messenger RNA (mRNA) isolated from various human tissues, CDNA sequences were amplified and the PCR products were sequenced using the dideoxy chain termination method on both strands.

Two distinct complete coding segments were identified by open reading frame analysis, each made up of a single exon encoded on the sense or antisense strands of genomic DNA. Proteins encoded by the new 21q22 tumor suppressor gene were then generated by conceptual amino acid translation of the predicted oligonucleotide sequences of SEQ ID NO: 4, SEQ ID NO: 5 and SEQ ID NO: 6 and are shown as SEQ ID NO: 1, SEQ ID NO: 2, and SEQ ID NO: 3. These amino acids sequences were then compared for sequence homology with known proteins using NCBI blastp program. The predicted amino acid sequences were also classified using the PIR-International Protein Family Classification System (Barker, et al., Nucleic Acids Res. 28:41-44 (2000); Huang et al., Nucleic Acids Res. 28:273-276 (2000)). Potential functional characteristics of the predicted protein were also determined by comparative analysis of the primary amino acid compositions using the TMHMM1.0 software package for the prediction of transmembrane helix formation in mammalian proteins (Sonnhammer et al., Ismb 6:175-82 (1998)). Structural prediction revealed that the 21q22 tumor suppressor gene encodes proteins which are secreted as growth control factors (particularly SEQ ID NO: 1) or which alternatively play a role as nuclear factors in cell growth control (SEQ ID NO: 2).

All references cited herein are fully incorporated by reference. Having now fully described the invention, it will be understood by those of skill in the art that the invention may be performed within a wide and equivalent range of conditions, parameters and the like, without affecting the spirit or scope of the invention or any embodiment thereof.

What is claimed is:

1. A substantially pure protein consisting essentially of the amino acid sequence of SEQ ID NO: 1.

2. A substantially pure polynucleotide consisting of nucleotides encoding the protein of claim 1.

3. An isolated polynucleotide consisting essentially of the nucleotide sequence of SEQ ID NO: 4.

4. A vector comprising the isolated polynucleotide of either claim 2 or claim 3.

5. A host cell transformed with a vector of claim 4.

6. The vector of claim 4, wherein said vector comprises a structural region consisting of nucleotides encoding the protein of claim 1 and which is operably linked to a promoter.

7. A host cell transformed with the vector of claim 6.

8. A substantially pure protein consisting essentially of the amino acid sequence of SEQ ID: 2.

9. An isolated polynucleotide consisting of nucleotides encoding the protein of claim 8.

10. An isolated polynucleotide consisting essentially of the nucleotide sequence of SEQ ID NO: 5.

11. A vector comprising the polynucleotide of either claim 9 or claim 10.

12. A host cell transformed with the vector of claim 11.

13. The vector of claim 11, wherein said vector comprises a structural region consisting of nucleotide encoding the protein of claim 8 and which is operably linked to a promoter.

14. A host cell transformed with the vector of claim 13.

15. A substantially pure peptide consisting essentially of the amino acid sequence of SEQ ID NO: 3.

16. An isolated polynucleotide consisting of nucleotides encoding the peptide of claim 15.

17. An isolated polynucleotide consisting essentially of the nucleotide sequence of SEQ ID NO: 6.

18. A vector comprising the polynucleotide of either claim 16 or claim 17.

19. A host cell transformed with the vector of claim 18.

20. The vector of claim 18, wherein said vector comprises a structural region consisting of nucleotides encoding the protein of claim 15 and which is operably linked to a promoter.

21. A host cell transformed with the vector of claim 20.

22. An assay for determining whether a subject has a predisposition to develop cancer comprising:
(a) obtaining a cell or tissue sample from said patient; and
(b) determining whether nucleic acid derived from said cell or tissue sample has a mutation that would affect the biological activity of a polypeptide selected from the group consisting of: SEQ ID NO: 1; SEQ ID NO: 2; and SEQ ID NO: 3.

23. The assay of claim 22, wherein said cancer is multiple myeloma.

24. A method of treating a patient for cancer, comprising transforming the cells of said patient with the vector of any one of claims 6, 13, or 20.

25. The method of claim 24, wherein said cancer is multiple myeloma.

26. A method of treating a patient for cancer, comprising administering to said patient the peptide of SEQ ID NO: 3 at a dosage sufficient to retard the growth of cancer cells.

27. The method of claim 26, wherein said cancer is multiple myeloma.
